(19) United States
(12) Patent Application Publication
(10) Pub. No.: US 2003/0013117 A1
(43) Pub. Date: Jan. 16, 2003

(54) Multiplexed differential displacement for nucleic acid determinations

(75) Inventors: Sharat Singh, San Jose, CA (US); Anita Inamdar, Sunnyvale, CA (US); Edwin F. Ullman, Atherton, CA (US); Liching Cao, Vallejo, CA (US); David Albagli, Millbrae, CA (US)

Correspondence Address: PERKINS COIE LLP, P.O. BOX 2168, MENLO PARK, CA, 94026, US

(73) Assignee: ACLARA BioSciences, Inc.

(21) Appl. No.: 10/245,030

(22) Filed: Sep. 16, 2002

Related U.S. Application Data
(63) Continuation of application No. 09/684,590, filed on Oct. 5, 2000.
Application No. 09/684,590 is a continuation-in-part of application No. 09/609,279, filed on Jun. 30, 2000.
Application No. 09/609,279 is a continuation-in-part of application No. 09/354,629, filed on Jul. 16, 1999.

(51) Int. Cl. C12Q 1/68; C07H 21/04

(52) U.S. Cl. 435/6; 536/25.4

(57) ABSTRACT

Multiplexed determinations of large numbers of events are achieved in an accurate and simple manner by using a multitude of primer reagents in combination with different capture reagents that serve for sequestering all the reagents at a single site, followed by independent release of subsets of the primer reagents using differential release conditions. Also included as part of the primer reagents may be identifiers, which serve to identify a particular characteristic. The method is illustrated using primers with sequences for initiation of chain extension that are joined to or serve as a capture sequence, and where the extended primer has an identifier. After extending the primer, the extended primers are sequestered via the capture sequence onto a sequestering agent, sequentially released and the released extended primers analyzed to provide multiplexed determinations. The subject method finds application for nucleic acid sequencing, single nucleotide polymorphism determinations, identification of nucleic acid fragments, and the like.

### CROSS REFERENCE TO RELATED APPLICATIONS

This application is a Continuation-in-Part of application Ser. No. 09/609,279, filed on Jun. 30, 2000, which is a Continuation-In-Part of application Ser. No. 09/354,629, filed on Jul. 16, 1999 which disclosures are incorporated herein by reference.

### INTRODUCTION

Background

Knowledge of nucleic acid sequences of species is increasingly important for many different purposes. The large number of bases in even the simplest genomes makes the sequencing of the genomes for the different species of interest a daunting task. Not only is there interest in a consensus sequence for a particular species, but there is also interest in determining the differences between individuals of a single species. There are many motivations for sequencing nucleic acids, including de novo sequencing, or resequencing for comparative, confirmatory or forensic applications and the like. In order to improve the rate at which nucleic acid sequences can be determined, automated machines have been devised, which primarily rely on the use of the polymerase chain reaction to amplify genomic DNA fragments, followed by primer extension and termination, using capillary electrophoresis for analysis. Using a single primer for the primer extension is slow, and inefficient in terms of throughput and reagent usage. Desirably, one would wish to have a plurality of different primers, e.g. multiplexed primers, spaced along a long strand of DNA or different strands of DNA, so that one could simultaneously sequence multiple kilobases. Multiplexing of this type is problematic without a method to separate the extended fragments from each of the regions, in order to be able to define the sequences.

In addition to sequencing there are many types of genetic analysis methods for which the means to multiplex would be of great benefit. For example, the possibility of using groups of single nucleotide polymorphisms (“SNPs”) to characterize or identify different traits has become an area of major interest. This area of interest is presently in the phase of cataloging SNPs. Once a sufficient number of SNPs have been identified for a statistically significant population, the major effort of relating SNP profiles to traits will begin. In each instance there will be a large number of nucleotide determinations to be made by, for example, single base primer extension or ligation reactions, which, if made individually will be very inefficient and expensive. Multiplexing provides greater efficiencies of throughput and cost because many reactions are run simultaneously in the same pool of reagents. However, the efforts to use multiplexing are confounded by the large number of DNA, RNA, or nucleotide molecules involved, the errors that inherently occur and the possibility of their amplification, and the impediments to separation of the sequences to obtain substantially pure fractions. Also, there is the desirability, to the extent it is possible, to recover and reuse reagents.

Although many methods for sequence-specific or primer-specific DNA purification methods have been described, including methods based on triplex affinity capture, peptide nucleic acid mediated capture, sequence affinity, or biotin capture, they do not address the means to perform the purification of multiplexed samples with independent release of the multiplex components.

There is, therefore, substantial interest in developing improved processes for performing genetic analyses using multiplexed protocols that provide for accuracy, efficiency in the use of time, equipment and reagents and reproducibility.

### BRIEF DESCRIPTION OF THE PRIOR ART

U.S. Pat. No. 5,648,213 discloses the use of strand displacement. U.S. Pat. Nos. 5,514,543 and 5,580,732 describe DNA sequence detection using multiple probes in a single assay. WO98/US33808 describes biopolymers attached to a support with a reversible link. WO98/14610 describes multiplex polynucleotide capture methods and compositions, in which the capture of the various primers occurs at distinct locations, and wherein the release conditions for the various primers are substantially the same. EP 0 416 817 describes primers containing polynucleotide tails.

### SUMMARY OF THE INVENTION

Methods and compositions are provided for separating a set of reagents into subsets by capturing the set and specific releasing each subset. The method is exemplified with nucleic acid reagents that are captured by hybridization and selectively released, using a sequestering agent and a plurality of pairs of nucleic acid reagents and capture reagents. Each nucleic acid reagent comprises a sequence part for hybridizing to a capture reagent, and the capture reagents comprise a complementary sequence to each of said sequence parts. Combining the nucleic acid reagents, capture reagents and sequestering agent provides for the capture of the reagents by the sequestering agent, which is then processed by sequentially increasing stepwise the stringency conditions to cause the sequential release of each subset of nucleic acid reagents, thus providing the separated subsets.

In one aspect, methods and compositions are provided for multiplexed determinations of at least one characteristic of a plurality of target moieties. In a single step, the plurality of moieties is processed to provide an assemblage of assay entities to be defined to provide the characteristic(s) of interest of the target moieties. The method is exemplified with nucleic acids as the target moieties, for sequencing, genotyping and the like. By using a plurality of different primers, modifying the primers, sequestering the (modified) primers, releasing the (modified) primers in portions using conditions for selective displacement and assaying each portion of the released (modified) primers, multiplexed determinations are performed for identifying characteristics of a target moiety. The compositions employed comprise sequestering supports having a plurality of capture probes for the capture of primers as a single population and the subsequent selective release of primer subsets, by use of media of varied stringency or reagents that alter the binding affinity of the primer/capture probe duplex. Also present may be labels, which further allow for differentiation of the different primers.

### BRIEF DESCRIPTION OF THE DRAWINGS

FIG. 1 shows multiplexed primers capable of differential release by capture sequence modification.

FIG. 2 shows multiplexed primers differentiated by identifier within sets differentiated by capture sequence.

### DESCRIPTION OF THE SPECIFIC EMBODIMENTS

Methods and compositions are disclosed for multiplexed determinations of target moieties. The methods and compositions employ nucleic acid sequences as primers and as capture probes for the capture of homologous nucleic acid sequences (e.g. primers). In performing the method, a mixture of primers specific for target nucleic acid sequences are combined with single-stranded target moieties under hybridization conditions, wherein primer/target duplexes are formed. The primers are then modified in various ways, depending on the characteristic of interest of the nucleic acid target moiety. The modified primers are released from the target moieties and captured onto a support using homologous nucleic acid sequences (capture probes) for sequestering the modified primers. The supports are optionally washed to remove unbound components.

The modified primers are all sequestered as one population. In one embodiment, the modified primers are sequestered as a random mixture on the support. In another embodiment the modified primers are separately sequestered at a plurality of sites within a common, fluidly connected region such that all sites are subject to the same conditions. In either embodiment, the mixture of modified primers is resolved by the selective release of primers from the support. Primers are released from capture probes into solution by the adjustment of a combination of the solution stringency, electric field and the duplex structure to overcome the binding affinity of the primer/capture probe hybrids. Each group of released extended primers may then be analyzed for the characteristic of interest.

The subject methods comprise identification of target species in a complex mixture, where a large number of different target species are of interest. The subject method finds particular application for nucleic acid sequencing, single nucleotide polymorphism (“SNP”) determinations, fragment identification, genotyping in association with cell strains, phenotypes, etc., allelic profiling and the like. Normally, these determinations are made in the presence of a large amount of nucleic acid, such as a cellular genome, cDNA transcripts from a cell, a complex mixture of DNA and RNA, and the like. Generally, the size of the DNA or RNA will be at least about 2 kb, more usually at least about 5 kb, and may be a full genomic complement. Within this amount of DNA or RNA only a fraction may be of interest, ranging from around one hundred per trillion bases in the case of SNP typing of human genomes, to up to around 50% in the case of sequencing plasmid inserts, or 90% or greater when sequencing PCR amplicons.

The reagents required are primer oligonucleotides and capture oligonucleotides, which are bound to or capable of being bound to a support. Each of these oligonucleotides will usually differ as to composition and function.

In referring to oligonucleotides, it is intended to include not only the naturally occurring nucleotides, but nucleotides which are functionally equivalent for the purposes of this invention. The nucleotides may be varied as to the backbone, the phosphate and sugar being replaced with equivalent moieties, such as phosphoramides, amino acids, phosphotriesters, methyl phosphonates, thiophosphates, thiophosphoramides, arabinosides, etc. Both natural and unnatural bases and sugars may be employed that provide the desired binding affinity with a homologous sequence.

For example, a number of unnatural bases are found to have higher binding affinity than the natural bases they replace, such as, for example 5-(1-propynyl)uracil, and 5-(1-propynyl)cytosine, which are described in U.S. Pat. No. 5,830,653. Also, some unnatural nucleotide structures have been shown to have higher binding affinity to nucleic acid sequences than a natural nucleotide structure, particularly peptide nucleic acids (PNA), where the phosphate ester backbone is substituted with a polyamide backbone (Nielsen, P. E., Egholm, M., Berg, R. H. and Buchardt, O. “Sequence-selective recognition of DNA by strand displacement with a thymine-substituted polyamide” Science 254 (1991) 1497-1500; Nielsen, P. E. et al., U.S. Pat. No. 5,539,082; Buchardt, O., Egholm, M., Nielsen, P. E. and Berg, R. H. “Peptide Nucleic Acids” PCT WO 92/20702 (1992); Thomson, S., Noble, S. and Ricca, D. “Peptide nucleic acids and the effect on genetic material” PCT Appl. WO 93/12129 (1993)). In particular, it may be advantageous to employ unnatural components to control the binding affinity of the primer and the capture oligonucleotide. Also, for reducing the likelihood of non-specific hybridization it may be advantageous to pairwise substitute bases in any pair of probes that are to bind to one another with moieties that preferentially bind to each other rather than to any of the natural bases. For example, iso-dC and iso-dG bind to one another but will not effectively base pair with adenosine, thymidine, cytidine, guanosine, or uridine, as described in U.S. Pat. No. 5,681,702.

The primer oligonucleotide will preferably have two nucleic acid sequence parts, and optionally a non-replicable moiety between, or a junction preventing polymerase activity across the two nucleic acid sequence parts. Primers of this general type have been described earlier, in EP 416817 and WO 94/21820, which are herein incorporated by reference. The first sequence part is a target binding sequence comprising at least about 8 nucleotides, more usually at least about 12 nucleotides and which may have 30 nucleotides or more. The greater the number of complementary nucleotides, the greater the specificity is for the target. The greater the complexity of the target composition, the more desirable it is to have a longer oligonucleotide. The primer oligonucleotide will also have a second sequence part designed to hybridize to the capture oligonucleotide, having at least 5 nucleotides, usually at least 6 nucleotides and may have as many as 80 or more, more usually not more than about 50 nucleotides. The considerations are the desired level of binding affinity, specificity, the costs of preparing the oligonucleotides, the level of complexity of the sample composition, and the like.

The second sequence part may be distinct from the first sequence part, identical to the first sequence part, or comprise some of the first sequence part. Usually, the oligonucleotide portions together will be at least 13 nucleotides, more usually at least about 18 nucleotides, and not more than about 100 nucleotides.

Where the two sequence parts are distinct, there may be an optional non-replicable moiety linking the two sequence parts of the primer oligonucleotide. This linking moiety is comprised of at least two atoms in the chain and not more than about 120 atoms in the chain, preferably not more than about 60 atoms in the chain, more preferably not more than about 30 atoms in the chain. A methylene chain may be used, such as propyl, dodecyl, octadecyl and the like. Where solubility is a consideration, the chain may also contain one or more heteroatoms, usually selected from nitrogen, oxygen, sulfur and phosphorus, although other atoms may also be present. Conveniently, the linking group may be an amino acid or polypeptide, a polyether such as polyoxyethylene, non-replicable nucleotides, a polyester, etc. Polyoxyethylenes such as di-, tri-, tetra-, penta-, hexaethyleneglycol and the like are particularly useful for their conformational flexibility and hydrophilicity. Generally, the linking group will be coupled to the oligonucleotide sequences via phosphodiester groups and the like, using standard coupling chemistry compatible with automated synthesizers.

Where the primer oligonucleotide is to be used in a polymerase chain reaction, a means to prevent the polymerase from extending into the capture sequence part of the oligonucleotide and rendering that part double stranded is desirable. A non-nucleosidic linking group incorporated between the two sequence parts is desirable for its ability to act as a PCR stop. The polymerase enzyme will not extend across the link because it is non-nucleosidic. Alternatively, a means to create a PCR stop without incorporating a linking group is to prepare the two contiguous sequence parts with reversed 5′-3′ orientation of the two sequences. In other applications, the linking group may be desirable for providing steric relief for the different hybridizing components.

The orientation of the first sequence part is determined by the enzymatic method that will be used to modify the primer. Where the primer is to be modified with a polymerase or a transcriptase, the 3′ end will be a free 3′-hydroxyl group, and the 5′ end will be joined with the second sequence part or optionally the linking moiety. Where the primer is to be used with a Cleavase enzyme, ligase, restriction endonuclease, and the like the structure of the primer first sequence part will be adapted to the requirements for enzymatic activity. For example, where a ligase is used, the primer first sequence part may be prepared with either a free 3′-hydroxyl group or a free 5′-phosphate, depending on the needs of the assay, ease of synthesis, etc., whereby the other end will be joined with the second sequence part.

The first sequence part will be substantially homologous, usually fully complementary to the target sequence. Usually, there will be less than 20% homology difference, more usually less than about 15% homology difference between the oligonucleotide sequence and the target sequence, as determined by such methods as described in Shpaer et al., Genomics 1996, 38: 179-91; States and Agarwal, Ismb 1996; 4:211-7; Mott et al., Comput Appl Biosci 1989, 5:123-31; and Pearson and Lipman, PNAS USA 1988, 85:2444-8, particularly the LFASTA method. For the most part there will be fewer than 5, more usually fewer than 3, preferably not more than about 1, nucleotide substitution, insertion or deletion, or combination thereof, between the first sequence part of the primer oligonucleotide and the target sequence.

The second sequence part need not be distinct from the first sequence part where the first part uniquely defines a subset within the plurality of primer oligonucleotides that is to be separated for analysis. Thus, when sequencing using unique primer sites, the primer oligonucleotide need only include the sequence homologous to the target sequence, where the same sequence will also serve as the second sequence part, i.e., to hybridize to the capture oligonucleotide. Whereas when performing genotyping analysis, each subset of primers to be separated for analysis will usually comprise a plurality of primers, and thus each primer will require a second sequence part to define the subset to which it belongs.

The orientation of the second sequence part is the same as that of the first sequence part where the two sequence parts are not distinct, i.e. overlap. The second sequence part may have the opposite orientation by design, where the opposite orientation provides a junction that acts as a polymerase stop. In most other cases the orientation may be different from that of the first sequence part, however for synthetic convenience the two sequence parts will often possess the same orientation.

The second sequence part of the primer oligonucleotide will generally be complementary to the capture oligonucleotide. Usually it will be desirable to have high affinity and specificity in the capture process. Generally, the portions of the primer oligonucleotide and capture oligonucleotide that bind to one another will be complementary and the sequence will be selected to provide unique binding in relation to other sequences which may be present during the capture stage.

A set of primer oligonucleotides is comprised of a plurality of primers, which can be divided into subsets wherein all primers having substantially similar second part sequences are members of the same subset. Thus, sets of primers may range from those in which all subsets have one member, e.g., each primer has a unique second part sequence, to those in which subsets are comprised of a plurality of members, wherein a plurality of primers of unique first part sequences share second part sequences that are substantially the same. In saying second sequence parts are substantially the same, it is meant that though not necessarily identical, they bind to the same capture oligonucleotide sequence, and are released under the same conditions.

The primers comprising a set are designed such that the primer subsets can be released separately, in stages, by changing the stringency of the solution contacting the support. At any point in the release process the subset of primers to be released has the lowest binding affinity and the stringency of the release buffer is such that the primer subset to be released is substantially denatured, whereas the other primer oligos remain substantially bound to the support.

The duplex melting temperature, or Tₘ, provides a useful basis for comparing the binding affinity of a series of duplexes. At its melting temperature, the strands of a duplex are in an equilibrium state where half the strands are hybridized and half are denatured. By incubating the solution at the appropriate temperature for a given solution composition, at least about 2° C., preferably at least 3° C. and maybe as high as 5° C. or greater above the Tₘ of the duplex with lowest binding affinity but below the Tₘ of the other bound duplexes, selective release is achieved. Preferably, the Tₘ of each primer subset, at the stage at which it is to be released, differs by at least 10° C. from that of any other bound primer subset. Although melting temperatures are a convenient means for comparing binding affinities in a given buffer solution, for the purpose of this invention, temperature is just one means for adjusting stringency, and other means such as salt concentration or organic solvent content may be more convenient.

The primer second part sequences may be designed to have the necessary binding affinity differences without modification of the oligonucleotide. By appropriate choice of the sequence and the structure, for example the number of bases, the A,T vs. G,C content, the presence of modified bases with higher or lower binding affinity, backbone modifications, PNA sequences and the like, the binding affinity of an oligonucleotide can be readily adjusted relative to that of another. The design of oligonucleotides with predetermined melting behavior is well known to those skilled in art. In this case the selective release of the (modified) primers occurs by manipulation of the solution conditions, such as temperature, ion concentrations, solvents, electric fields, and the like.

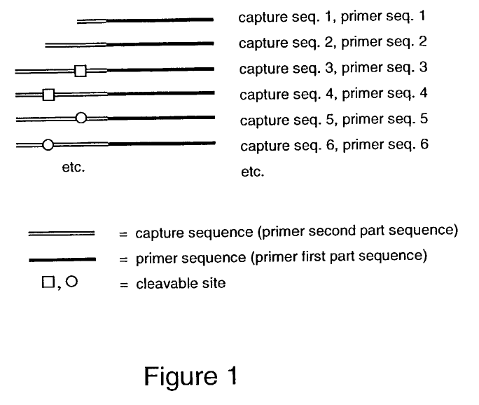

The primer second part sequences may also be modified to alter the original binding affinity. In this case primer modification is employed as a variable for effecting the selective release of a primer subset. For example, the modification may comprise cleaving the second part sequence into smaller fragments whereby the binding affinity will be decreased accordingly. A primer set designed for selective release by both stringency modulation and primer modification is illustrated in FIG. 1.

In FIG. 1, the strength of the binding affinity is indicated by the length of the capture sequence for illustrative purposes only. Capture sequences may be designed that are similar in length but differ with respect to G,C content or the number and nature of base modifications that result in sufficiently different binding affinities. Thus, first capture sequence 1 and capture sequence 2 would be selectively released by introducing release buffers with the requisite stringency to yield the selective and separate release of each primer. The remaining primers would then have similar, high binding affinities but may be distinguished by cleaving portions of the capture sequence to reduce the binding affinity of at least one primer below that of the other unmodified primers such that selective release conditions can be established as described above. More than one primer may contain the same cleavage site, e.g. capture sequence 3 and 4 of FIG. 1, in which case the cleavage reaction would produce a series of primers with distinct binding affinities capable of selective release. Following the selective release of capture sequences 3 and 4, the next modification reaction may be performed to expose, e.g. capture sequences 5 and 6, the next set of primer subsets with distinct binding affinities that are subject to separate release by selective melting via stringency control.

The probe set of FIG. 1 is not intended to be a limiting example of a primer set. The number of primer subsets releasable by varying the stringency may be as many as ten, more usually about four, but may be as few as two. The number of primer subsets subject to the same modification reaction and which are then released by varying the stringency may also be as many as ten, more usually about four, but may be as few as two. The number of different modification reactions is not generally limited, except where matters of sensitivity, binding capacity, release efficiency, etc. are of concern.

The primer modification reaction may be an enzymatic, chemical, electrochemical or photochemical reaction. Restriction enzymes can be used to produce nicks in one strand of a duplex under partial digestion conditions or by substituting the phosphate group at the cleavage site of one strand, with e.g. phosphorodithioate, to render it refractive to the enzyme. The action of restriction enzymes are sequence specific, and therefore applicable to the generation of sequence fragments of predetermined length. Enzyme recognition sites may be designed into the sequence at desired locations. Other enzymatic reactions may be used for modifying the binding affinity of the primer second part sequences. RNase H digests RNA bases in DNA-RNA hybrids. Thus ribonucleotides may be included in primer second part sequences from the desired cleavage site extending out to up to the rest of the sequence that is to be cleaved away. Other enzymes such as, for example, carboxyesterases may also be employed for cleaving esters incorporated at a desired cleavage site in the primer second part sequence. Other enzymes that do not degrade DNA may also find use.

Alternatively, chemically reactive sites may be incorporated at desired locations in the primer. These sites do not react under normal handling, storage or hybridization conditions, but are chosen for their reactivity with a specific reagent that does not substantially react with other components that are present. For example, the cleavable site may be a disulfide, which may be cleaved by mild reducing conditions that also do not affect DNA. The cleavable site may be a photoreactive group activated by wavelengths not absorbed by DNA, or a metal complex that labilizes upon redox change.

In cases where the primer modification reaction occurs at a reactive site not naturally present in a DNA or RNA moiety, that reactive site is incorporated into the oligonucleotide backbone within the sequence. The reactive site is contained within a moiety that may be incorporating during oligonucleotide synthesis according to standard automated or manual synthetic methods. The reactive site-containing moiety may replace a sugar moiety or a phosphate diester moiety of the natural structure, and a nucleobase may or may not be joined to the reactive site-containing moiety.

There may be situations where one wishes to have a single capture oligonucleotide generally not more than about 5 kb, usually not more than about 2 kb, yet where differential release may still be obtained. In this situation, one could synthesize a single capture nucleic acid and several primer oligonucleotides, each of which has a second part for binding to different regions of the capture oligonucleotide. By appropriately spacing capture sequences complementary to the second part of each primer oligonucleotide on the capture oligonucleotide, one could employ a set of primer oligonucleotides that are capable of binding to the same capture oligonucleotide but to different regions such that the desired level of independent release is possible by stringency control. Generally one would wish to have fewer than about 10 different primers captured by a single capture oligonucleotide, frequently fewer than 5 different primers, although depending on various considerations such as economics, specificity, sample size and complexity, one could have a single capture oligonucleotide for more than 10 primer oligonucleotides.

The capture oligonucleotide is comprised of a capture sequence(s), which binds to the second sequence part of a primer oligonucleotide. Generally, the capture oligonucleotide will be at least about 8 nucleotides and not more than about 100 nucleotides, usually not more than about 50 nucleotides. A capture sequence of the capture oligonucleotide may have the same length as the second sequence part of the primer oligonucleotide, although in some instances it may be different, usually not more than about 5 bases different.

In situations where one wishes to have a single capture nucleic acid, a plurality of capture sequences for binding the primer oligonucleotide may be interspersed with non-hybridizing sequences. By providing such non-binding sequences between each capture sequence one prevents primer oligonucleotides from influencing the binding affinities and dissociation rates of its neighbors, thus maintaining the ability to induce the independent release of each captured primer oligonucleotide.

Just as one may modify the second sequence part of the primer to alter the binding affinity of the primer oligo/capture oligo duplex, one may alternatively modify the capture oligonucleotide in the same manner as described above for the primer second part sequence. Where strand modification is used to control the selective release, use of either strand is equally preferred except where reuse of the capture oligonucleotide is desired, in which case incorporating reactive sites into the primer oligo is required.

The capture oligonucleotide may also comprise an ionic moiety or moieties. The moieties may be incorporated as modified nucleotides, or as non-nucleosidic components conjugated to internal or terminal bases, sugars or phosphate groups. Particularly useful are polycationic moieties, such as poly-(amino acids), such as poly-lysine, which may be conveniently coupled to the oligonucleotide, with appropriate modifications via disulfide bond formation or a sulfhydryl-maleimide coupling reaction. Methods for preparing oligonucleotide conjugates are well known. Or, a nucleoside derivative such as an amine-bearing derivative of thymidine may be conveniently incorporated using standard oligonucleotide synthetic methods. The ionic moieties may function to increase the local ionic strength around the hybrids formed in carrying out the subject invention, allowing the concentration of salts in the various buffers to be reduced without adversely affecting the duplex binding affinities and the ability to selectively release the bound multiplex of primers. This is particularly desirable where the released primers are to be analyzed by electrophoresis utilizing an electrokinetic injection, and the presence of excess salts would decrease the efficiency of the injection.

The capture oligonucleotide will be bound either directly or indirectly to a sequestering agent. Sequestering agents may include container walls, disks, porous or solid beads, fibers, capillary surfaces, polymers, dendritic materials and the like, in effect, any entity which allows for physical separation of what is bound to the entity and what is unbound, and also allows for washing to remove non-specifically bound compounds while retaining specifically bound compounds. Conveniently, the sequestering agent, or equivalently, the support, may be a bead or a spatially defined region of a container, well, or channel.

Usually, a linking group will be employed between the capture oligonucleotide and the support, generally a hydrophilic linking group, conveniently of at least two atoms in the chain and not more than about 120 atoms in the chain, preferably not more than about 60 atoms in the chain, more preferably not more than about 30 atoms in the chain. A methylene chain may be used, such as propyl, dodecyl, octadecyl and the like. Where solubility is a consideration, the chain may also contain one or more heteroatoms, usually selected from nitrogen, oxygen, sulfur and phosphorus, although other atoms may also be present. Conveniently, the linking group may be an amino acid or polypeptide, a polyether such as polyoxyethylene, an ester or a polyester, such as polyglycolide, etc. Polyoxyethylenes such as di-, tri-, tetra-, penta-, hexaethyleneglycol and the like are particularly useful for their conformational flexibility and hydrophilicity.

The chemistry for binding an oligonucleotide to a solid support is well known. The capture oligonucleotide or the linking group may be provided with a chemical reactant or one member of a binding pair suitable for reaction with or binding to an appropriately functionalized support. These methods are known in the art, and include for example biotin/streptavidin binding, or thiol/maleimide adduct formation. It may also be advantageous to employ multiple layers of linking groups as means of increasing the binding capacity, the surface coverage of the binding region, and the like. Multiple layers of linking groups are formed by successively treating the support with the appropriate molecules containing at least more than one reactant or binding site. For example, a streptavidin-coated support may first be treated with a poly-biotinylated molecule to produce a biotinylated support, which may then be treated with streptavidin to produce a support with exposed biotin-binding sites. This treatment may be continued with the biotinylated molecules and streptavidin the desired number of times to produce multiple layers of linking groups with the outer surface exposing the necessary binding pair member for immobilization of the capture oligonucleotide.

The capture oligonucleotide will typically be sequestered onto the support prior to contact with the sample solution containing the primer oligonucleotide. However, depending on the type of binding chemistry used, it may be convenient for the capture oligonucleotide and the primer oligonucleotide to be combined first, under hybridization conditions, to permit duplex formation. Following this, the duplex is contacted with the support where the capture oligonucleotide, appropriately modified as described above, reacts with or binds to an appropriately functionalized support. The order of combining the reagents may be varied accordingly, but wherein one forms a structure in which the primer oligonucleotide is reversibly bound to a support via the capture oligonucleotide.

A bead support may be of any convenient composition, such as latex, metal sol, colloidal carbon, polyacrylamide, etc., generally of a diameter in the range of about 1 μm to 1 mm, usually at least about 10 μm, more usually in the range of about 50 μm to 500 μm. The beads may be non-magnetic, diamagnetic or superparamagnetic, depending upon the mode of separation desired. A wide variety of beads are commercially available from different sources. The beads may be functionalized for linking the capture oligonucleotide or may have reactive functionalities for bonding the linking group. If the support is a bead, fiber, membrane or soluble polymer, it may be further linked to a solid surface such as a container wall, a larger bead or an insoluble polymer to facilitate the manipulation of said support. See, for example, U.S. Pat. No. 5,900,481, and references cited therein for a description of beads and conjugation of nucleotides to the beads.

The support may be a surface, which may be of any convenient composition, such as plastic, glass, silica, which in turn may be coated with polymers, biopolymers, or other molecules. The coating functions to reduce non-specific adsorption of the analyte or contaminants introduced by the sample itself. The coating may also function in the immobilization of the capture oligonucleotide to the support by providing a chemical reactant or one member of a binding pair with which the capture oligonucleotide may react. The coating may also comprise polyionic compounds, particularly polycations, such as polylysine or aminated dextrans, which may be included for regulating the ionic strength around the oligonucleotides. The support may also be a porous surface, such as a membrane, e.g. nitrocellulose.

The number of capture oligonucleotides of the same type that are bonded to an individual support will usually be at least about 10, preferably at least about 50 and may be 10⁸ or more, depending on the number of different beads or polymer supports necessary for the assay, the redundancy permitted, the multiplicity of targets, the sensitivity with which the different labels may be distinguished, and the like.

In addition, one may include with the primer oligonucleotide an additional region, which is referred to as an identifier, which during the primer modification process is present and remains part of the modified primer. Depending on the nature of the characterization, the identifier may serve to identify the presence of a particular nucleotide, the composition or identity of the primer, or provide other information of interest about the target nucleic acid. For example, in sequencing and SNP determinations, one may be interested in identifying the particular nucleotide at the terminus of the extended primer without having to uniquely label the terminating nucleotide. More particularly, when performing SNP determinations, i.e. genotyping of individual positions, by single base primer extension using methods such as those described in U.S. Pat. Nos. 4,656,127 and 5,888,819, the identifier may be a variable length of nucleotides not integral to the capture sequence or primer sequence portion of the oligonucleotide. Each different primer would be associated with a different identifier. To further increase the capability to multiplex, a similar series of variable length identifiers may be associated with different capture sequences.

In order to avoid sequencing the primer or where variation in mobility of different primers does not provide a sufficiently discrete opportunity for differentiation of the number of target sequences, the identifier may provide for a detectable label on the primer sequence.

The identifier label may take many forms. Depending on the method of detection of the modified primers, there may be an identifying label or no label. With separation methods, by having modified primers, which can be detected by differential mobility, e.g. chromatography, or electrophoresis, one may be able to detect the modified primer solely by its mobility. Identifiers of this type may be referred to as mobility tags. Such modified primers may also be separable based on differential mass to charge ratios, e.g. by mass spectrometry. For the same and other techniques, one may require a label, which provides for detection by electromagnetic means, e.g. fluorescence or electron ionization. For example, the identifier may comprise a labeled nucleotide, which is capable of being joined to or included in a growing nucleic acid chain and has a label, which allows for differentiation, such as different fluorescers, electrophoretic tags, which allow for mobility discrimination, electrochemical tags, chemiluminescent tags, gas chromatographic tags, etc. or physical separation, such as ligand-receptor combinations.

Similar labels may be bound to the primer, but fluorescers will usually be of limited diversity. Where the primer varies as to mobility, the diversity will be expanded by the number of different fluorescers, which one may detect and accurately distinguish. Usually, one does not wish to have more than about two different excitation sources, which greatly narrows the multiplicity achieved with fluorescent labels. However, fluorescent semiconductor nanoparticles, such as those described in Science (1998) 281:2013-2016, may be of use as fluorescent labels with tunable, narrow emission bands with broad, matched excitation bands. Also, four-color fluorescent tag sets have been developed for DNA sequencing applications.

For mobility tags, one may use oligomers, such as peptides, oligonucleotides, organic oligomers, such as polyethers, polyesters, and polyamides, polyhaloalkanes, or substituents other than halo, such as cyano, oxy, thio, nitro, and the like. By virtue of the subject invention, using combinations of differentiation characteristics, a very large number of different attributes can be imparted to the primer and identifier, so as to permit a very large number of target sequences to be addressed in one or a few vessels.

In carrying out the method, the target species will be single stranded nucleic acid, DNA or RNA. The nucleic acid may come from any convenient source, prokaryotic or eukaryotic genomes, cDNA from prokaryotic and eukaryotic sources, mitochondrial DNA, rRNA, mRNA, synthetic DNA plasmids, cosmids, YACs, viruses, and the like. Where the DNA is double stranded, it will be denatured to provide single stranded DNA. The DNA may be further processed by mechanical fragmentation or restriction enzyme digestion. Conveniently, the fragments may be less than about 1 centiMorgan, usually less than about 10⁵ nucleotides. The target nucleic acid is combined with the primer oligonucleotide under nucleic acid modification conditions, usually extension or restriction conditions.

The method employs as a modifying reagent system, besides the primers for each target nucleic acid, an enzyme having polymerase activity, which may also have 5′-3′ nuclease activity, e.g. Klenow fragment of DNA polymerase, DNA polymerase 1, Taq polymerase, etc., an enzyme having 5′ nuclease activity such as Cleavase®, ligase, restriction endonuclease, nuclease or transcriptase activity. Accordingly, for primer modification the modifying reagent system may involve amplification, sequencing, mini-sequencing, SNP determination, strand cleavage, ligation, restriction, transcription or other purpose, which involves an interest in characterizing a plurality of target nucleic acid sequences. Usually, there will also be amplification of the target nucleic acid, performed in accordance with conventional methods, adding dNTPs and thermal cycling, as required, whereby the primer is extended. The thermal cycling involves a lower temperature step of extending the primer and a higher temperature step of denaturing the resulting duplex, followed by cooling to allow for hybridization of unextended primer to target in preparation for another step of primer extension. For methods of performing nucleic acid extensions using a polymerase, see, for example, PCR Methods and Applications (Cold Spring Harbor Laboratory Press, Cold Spring Harbor, N.Y, 1994); Pastinen, Clin. Chem. (1996) 42:1391; U.S. Pat. Nos. 4,683,195, 4,683,202, 4,800,159, 4,965,188 and 5,008,182.

The particular method for performing the nucleic acid modification is not critical to this invention and any of a variety of ways may be employed, which may additionally involve various agents associated with the characteristic of interest, such as labeled terminators, labeled dNTPs, labeled ddNTPs, Cleavase®, ligases, nickases, restriction endonucleases, RNA, etc. See, for example, U.S. Pat. No. 5,422,253 and U.S. Pat. No. 5,712,124.

For sequencing and SNP determinations, as well as other assays where one wishes to know the particular nucleotide which has been added, either a labeled terminating nucleotide is employed or a labeled primer is employed, but in the latter case, four reaction vessels are used, each with a different terminator associated with a specific fluorescent labeled primer. Thus, each of the four terminating nucleotides would be associated with a different label, which would allow for their differentiation. Conveniently, fluorescent labels are used, such as FAM, ROX, TAMRA, TET, JOE and the like, or the “BIG” fluorescers are used, where fluorescein is bonded to another fluorescer, such as FAM, ROX, TAMRA, TET, JOE and other rhodamine or dichlororhodamine derivatives, and the resulting compounds are referred to as energy transfer dyes. Another family of energy transfer dye sets incorporates cyanine as the primary energy donor. Dyes which may be used are described in U.S. Pat. Nos. 4,997,928 and 4,855,225 and PCT application Nos. US90/06608 and US90/05565. The terminator may be any molecule that is recognized by the polymerase being used, specifically binds to the complementary nucleotide present in the target, and cannot be extended. Thus, various modified or capped nucleotide analogs may be employed, but to the extent that the dideoxynucleotides are readily available, come with a variety of labels, and the conditions of their use are well established, they are the terminators of choice. However, there exist applications for which four distinct labels for the identification of A, T, C or G are not required or are not available. For example, two-color sequencing is practiced and kits are available.

For sequencing, the subject invention permits sequencing reactions of a plurality of target sequences to be performed in a multiplexed manner, therefore affording more efficient use of reagents, resources and time. The targets to sequence could derive from one contiguous strand that could be 1 kb or more bases in length. Alternatively, the targets for sequencing could derive from a plurality of strand fragments, plasmids or vectors. For sequencing long portions of any one strand, depending on the fidelity of the system and the capabilities of the analysis method, the primers would be spaced about 0.5 kb apart, desirably about 0.8 kb apart and even further, if the system permits accurate resolution at such spacing.

A plurality of primer oligonucleotides are used for the sequencing, wherein each primer first sequence part is uniquely associated with a second sequence part, however more than one identifier may be associated with each primer oligonucleotide. The sequencing reaction may be performed on the plurality of targets simultaneously, or in separate vessels according to the needs for associating distinct identifiers or capture sequences with the different targets. For example, if each of the targets require unique sequencing primers then where each of the terminators is differently labeled, unique primer oligonucleotides can be employed in the same vessel. Thus the multiple target sequencing reactions are performed at the same time in the same reagent system and the extended primers are then separated for analysis. The entire set of primers is captured and then primers with the same second sequence parts are selectively and separately released at each stage of the release process and analyzed.

Alternatively, separate vessels may be employed in sequencing reactions where labeled primers are desired rather than labeled terminators. In this case, as is typically done where labeled primers are used, there are two to four separate reactions carried out using primers that comprise the same first and second sequence parts but have different identifiers. Thus, a plurality of primers are divided into two to four groups where the primers in each group are labeled differently from their same primer in any other group. The termination reactions are defined to be different in each vessel, therefore associating a different terminating base with each different identifier. Multiplexed sequencing reactions are performed in the separate vessels, and the primers are then combined and finally separated for analysis. In the subject method, the separation occurs by differential strand release based on the differences of the binding affinity of the primers, more specifically the primer second sequence parts. At each stage of the release process, the primers from the different reaction vessels but with common second sequence parts are released and analyzed. The subject invention allows one to manipulate, separate or combine for analysis the different primer extension products from the different sets and thus to perform sequencing analysis on multiplexed sequencing reactions.

For genotyping single base positions to identify SNPs, triplet deletions or insertions using single base primer extension methods using a plurality of primer oligonucleotides, the targets may be present on a contiguous nucleic acid strand, or derive from a plurality of strands, strand fragments, chromosomes, plasmids or genomes, etc. Moreover, to enhance the specificity of the genotype determination at each position, both the sense and antisense strands present in the sample may be interrogated by primer oligonucleotides targeting each strand.

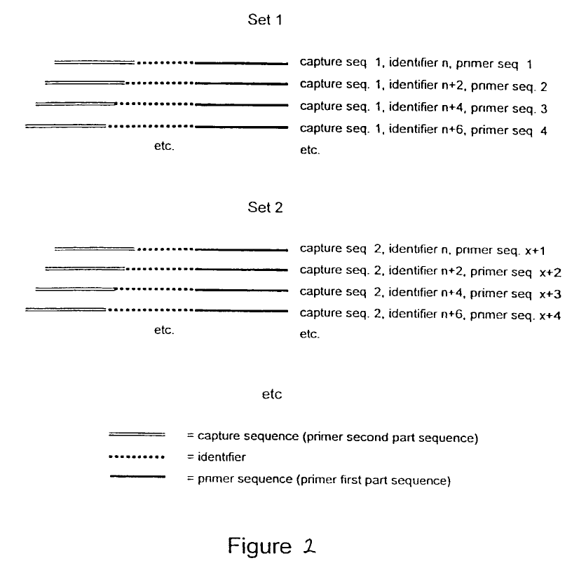

An exemplification of a primer reagent with identifiers that act as mobility tags for multiplexed genotype determination is shown in FIG. 2. The genotype is determined by modifying the primer oligonucleotides in a single base extension reaction. The primers hybridize to the homologous target sequence adjacent to the position of interest. In the presence of a polymerase enzyme and terminating derivatives of at least one of the four nucleotides, the enzyme adds to the end of the primer the base complementary to the base found in the next position on the target. The new base at the terminus of the primer cannot be extended, thus each primer increases in length by one base.

The primer reagent is comprised of a plurality of primer oligonucleotides that are divided into subsets, and within each subset the primer oligonucleotides have the same primer second part sequence (capture sequence) homologous to a capture oligonucleotide.

Associated with each primer first part sequence is an identifier, which only need be unique within each subset. The same identifiers may be used in the different subsets. The identifier may be comprised of a sequence of n units, n being at least 1, and usually not more than about 20, but can be as many as 50 units. For convenience, the unit will be a nucleotide base, which can be incorporated into the primer oligonucleotides by standard automated DNA synthetic techniques. The primer first part sequences are conveniently designed to be of equal length, so that the overall length of the oligonucleotides, and therefore the mobilities, are differentiated by the length of the identifiers.

The length of the identifier is primarily chosen for convenience in the preparation of the primer oligonucleotides and for the separation and detection of the released primers. Each identifier may differ in length by at least one base, because single base resolution is normally achieved by common mobility-based assay methods, i.e. electrophoresis. However, the identifiers may differ by two base units to facilitate detection and to avoid having both modified and unmodified primers of similar length.

The number of identifiers determines the number of primer oligonucleotides within each subset. In FIG. 2, x represents the number of such identifiers within a subset. The number of subsets, multiplied by the number of identifiers in a subset is the total number of multiplexed determinations.

For greater specificity in the genotype determination, the same type of analysis may be performed on both strands of a double stranded target. Thus, both the base and its complement are determined for each position of interest. There are many ways to design primer oligonucleotides for the analysis. For example, each set of primer oligonucleotides (cf. FIG. 2) may be dedicated to one strand of the target, or each set may contain the probes for analyzing both strands for given group of positions of interest.

As previously indicated, the amplification will normally depend upon extending the primer with a polymerase, separating the extended primer from the target sequence, which normally involves thermal denaturation, recreating hybridization conditions, where unextended primer will hybridize to available sites on the target, and repeating the extension. This process may be repeated a sufficient number of times to provide the desired amount of extended primer. Depending on the nature of the extension reaction, duplexes may have to be denatured to provide the single stranded extended primers. Once the primer modification has been performed, the modified primer oligonucleotides may then be harvested.

One may wish to separate modified primers from unmodified primers and other DNA present in the mixture. While this will normally not be necessary, such separation can be achieved for example, where the primer is modified by extension, by having a binding-member-labeled terminator, where the binding member has a complementary binding member bound to a support and is capable of ready release. By combining the reaction mixture with the support, only extended primers terminated with the binding member labeled terminator will be captured, and unbound and non-specifically bound DNA washed away. The captured extended primers may then be released. This secondary binding and release process may occur prior to or after the multiplexed strand capture and release process, preferably prior. While there are many different specific binding pairs that may be used, particularly convenient is the use of desthiobiotin and streptavidin, with biotin addition causing release by displacement. Other ligand-receptor pairs include: digoxin-antidigoxin; fluorescein-antifluorescein; saccharides and lectins, substrates/inhibitors and enzymes; etc.

Conversely, where the primer is modified by restriction a binding-member label may be used to separate the modified and unmodified primer oligonucleotides. By locating the binding-member label in the portion of the primer cut away from the portion containing the capture sequence and, if present, the identifier, then unmodified primer as well as the modification reaction side product can be separated away from the reaction product. The specific binding pairs described above may be used.

The number of capture sequences is related to the complexity of the sample, the binding capacity of the support, the degree of multiplexing, the resolution and sensitivity of the detection system and the number of different selective release conditions that may be achieved. The number of different capture sequences usually will be at least about 3, more usually at least about 4, usually not more than about 7, and may be 12 or more where primer modification reactions are used in the method.

In one embodiment, where the (modified) primers are sequestered as one population, the capture oligonucleotides are attached to the sequestering agent in a randomized fashion. A mixture of the oligos is prepared and contacted with the sequestering agent for binding or reaction, thus the capture oligos are all effectively located at one site. In another embodiment, where the (modified) primers are separately sequestered at a plurality of sites within a common, fluidly connected region, the capture oligos usually will be separately introduced for binding or reaction with a sequestering agent, such as a bead, or at a certain location on a sequestering agent, such as a container wall or channel. Preparing the capture oligos on a support in a stepwise fashion provides flexibility and convenience where large numbers of capture oligos are needed, or certain sequences are used interchangeably, and also simplifies assaying the quality of the immobilization procedure. By combining such sequestering agents within a common, fluidly connected region the capture oligo set is also effectively located at one site.

Within each group of (modified) primers there will usually be at least about 4 members, frequently at least about 10 members and there may be 1,000 or more, however in the case of genotyping by single base extension reactions there will be 2 members. For example, if one is doing sequencing of a large DNA sample and one can distinguish 600 different extended sequences reliably, then the number of bases that can be sequenced would be approximately the number of primers multiplied by 600. With a set comprising 6 primers, one would sequence about 3600 bases, with each primer having a different capture sequence and each primer would be associated with about 600 different extended sequences (members). In the case of SNPs, if one were interested in 2,500 SNPs and one had 100 distinguishable identifiers, then one would have 2,500 primer first part sequences with 25 capture sequences, with each capture sequence associated with 100 different identifiers and first part sequences. Or, using the same number of identifiers and capture sequences one could analyze both strands in determining 1,250 SNPs. Thus the number of distinguishable tags determines the size of each group that can be analyzed, and the number of different capture sequences associated with the primers, i.e. the number of subsets, is determined by the complexity of the target divided by the group size. The number of modified primers will be determined by how many different events can be associated with a specific primer, varying from 1 in the case of SNPs to 600 or more in the case of sequencing The reaction mixture, either processed, or without any processing, is combined with the capture oligonucleotide under hybridization conditions. Usually, stringent conditions will be used, the degree of stringency depending upon the multiplicity of sequences, the length of the sequences, the Tₘ of the sequences, etc. Stringency may be achieved by variation in salt (buffer) concentrations, solvents, temperature and the like. The choice of stringency will be determined by the ability to specifically distinguish between the individual primers present in the extended primer mixture. Generally, the density of capture oligonucleotides bound on the support will be in the range of 10⁵ to 10¹⁵ per mm², more typically about 10⁸ to 10¹² per mm², depending on the type of support, the desired concentration, the number of different sequences to be determined, and the like, where the ratio of capture sequences to primer sequences will be at least about 2:1 and preferably at least about 5:1, usually not exceeding about 10²:1.

Various wash and reaction buffers may be employed for reactions and washes. Buffers include saline, phosphate, carbonate, HEPES, MOS, Tris, TE, etc. Generally the buffer concentrations will be in the range of about 1 to 500 mM, more usually in the range of about 5 to 200 mM. The use of the individual buffers is conventional and a particular buffer will be used in accordance with the particular application. In some cases, wash buffers will contain a minimal salt concentration or no salt whatsoever. Low salt wash solutions are particularly desirable just prior to release of the captured primer for use in, for example electrokinetic transport, such as electrokinetic injection for capillary electrophoresis applications, or transfer into a mass spectrometer for analysis.

After a reaction, a portion of the primer oligonucleotides will have been converted to a mixture of modified primer products. The primers and sequestering agent will be combined and incubated for sufficient time for hybridization between the primers and the capture oligonucleotides under the appropriate stringency conditions. The sequestering agent may then be separated from the liquid medium, using physical separation, centrifugation, filtration, magnetic separation, etc. and the washed with an appropriate buffer. The separation of the liquid medium from the sequestering agent may take the form of flowing a buffer into the support area while forcing the original liquid out through an exit. The sequestering agent, freed of non-specifically bound DNA, excess salts, templates, target, monomers, enzymes etc. will then be combined with an appropriate buffer, or for beads, redispersed in an appropriate buffer, in preparation for release of the captured primers.

The stringency conditions for release will be selected to provide a high degree of specificity for the displacement. The stringency of the conditions will depend to a degree on the nature and length of the capture sequences, the Tm of these sequences, the variation in primer sequences, and the like. Generally, salt concentrations for release will be in the range of about 0.01 mM to 100 mM, temperatures will be in the range of about 20° to 90° C., more usually about 30° to 80° C., and while solvents other than water may be present, they will usually not be required, and if present, will generally be present in less than about 50% by volume. Solvents of use are those that are miscible with aqueous buffered solutions and do not precipitate oligonucleotides but have a denaturing effect, and include lower molecular weight amides, especially formamide, which is a common cosolvent for adjusting the solution stringency towards nucleic acids, or ethylene glycol. Salts may be included at concentrations of about 1 mM to 1 M that have a denaturing effect on nucleic acid duplexes, such as sodium perchlorate, tetramethylammonium chloride, or solutes such as urea at concentrations of up to about 4 M. The pH of the solution may be varied to effect strand release, typically varied by rendering the solution more basic. Also, an electric field may be used to induce the differential release of the primers. Methods have been described in U.S. Pat. Nos. 6,068,818; 6,017,696 and 5,849,486, which are herein incorporated by reference, for such electric field-induced effects applied to, for example, single base mismatch discrimination. In this case the support used for sequestering of the reagents must be located in the vicinity of an electrode, and at least one more electrode must be exposed to the solution to provide a circuit for control of an electric field. Obviously, the different parameters will be chosen to obtain the desired discrimination for strand release.

In one method of carrying out the differential strand release, conditions will be brought to desired levels of stringency in stages. Incubation will be performed for sufficient time for a substantial proportion of the modified primers having the sequence of lowest binding affinity to be displaced. Once the modified primers have been released, they may then be harvested and processed. One may continue increasing the stringency of the conditions and incubating the captured primers in a similar manner to induce the displacement of up to the rest of the extended primers. Preferably, the strand displacement conditions will be increased in stages, stepwise, releasing one primer subset at a time from the support to provide for the separate release of the primers.

The series of release conditions useful for each stage may be achieved by varying one or more parameters that determines the stringency of the conditions. For example, temperature or electric field strength may be varied using a given buffer system to release each of the primers. However it may also be desirable to vary the ionic strength or the organic solvent content of the buffer, or both, such that the incubation temperature is the same for each release, or so that temperature steps are smaller. With only small temperature increments for each release stage the overall operating temperature range is narrow, and more primer subsets can be employed and released within the overall operating temperature range.

In another method, in combination with stepwise increases in the stringency of the release solution, reagents are introduced for selective modification of the capture sequence of either the primer or the capture oligonucleotide. Incubation of the reagents and captured primers is carried out under conditions that permit the reaction to proceed but do not cause any of the primers to be released. After sufficient reaction period the reagents may be removed, the support optionally washed, and then treated with a release buffer, wherein the selective release of subsets of primers proceeds as described above. Such a series of modification reactions and selective release steps may continue until all of the primers have been released.

The release of the extended primers may take the form of having beads in a well having a membrane bottom and incubating the displacement strand with the beads for sufficient time for the appropriate group of extended primers to be released. Where the container has a permeable base, after sufficient time for displacement to occur, a differential pressure is created across the permeable membrane, so that the liquid containing the released extended primers is separated from the beads. Alternatively, the force to drive the liquid through the membrane may be generated by centrifugation. The liquid is isolated and then used for the next stage. For each displacement, the process is repeated, until all of the desired different extended primers have been substantially released. The beads may be washed between the application of the different stringency conditions. Alternatively, one could use a column in which beads are packed in substantially the same way as the container with the permeable membrane bottom. Another way is to incubate the support-bound primers with agitation, centrifuge to pellet the support beads and then draw off the supernatant. Or, if the beads contain a magnetic core, they may be pelleted by application of a magnetic field to aid in drawing off the supernatant liquid. Again the process is repeated with each desired strand displacement condition. Instead of beads one could have a capillary with the capture sequences conjugated to the wall of the capillary. Alternatively, one could have a flow channel in a planar substrate with the capture sequence conjugated to a surface of the channel, to beads contained within a section of a channel, or to a support confined within a channel. Other sequestering agents also find use, as appropriate.

Depending on the nature of the released extended primers, they may be isolated and processed in a variety of ways. Where the mobilities of the different extended primers are different, one may separate the extended primers by the different mobilities, depending on a detectable label for detection, when required. Separation can be achieved by electrophoresis, chromatography, gas or liquid, mass spectrometry, or the like. For electrophoresis and chromatography, a fluorescent tag can be provided on the primer or the terminator for detection of the individual bands of modified primers. Conventional conditions are employed for the separations. See recent reviews, for example, Mol. Pathol. (1999) 52:117-124.

The subject method is applicable where large numbers of determinations are of interest. These include sequencing of nucleic acids, detection of SNPs, identification of nucleic-acid fragments, and the like. The number of individual characteristics of interest will be at least about 10, more usually at least about 50, generally at least about 500, and may be 10,000 or more. For the most part, one vessel will be used, but in some instances, one may divide the determination into 4 vessels, one for each terminating nucleotide, or a multitude of vessels where similar cloning vectors may be uniquely addressed, so that a physical separation will contribute to the multiplexed diversity. In sequencing, for example, one could add the same family of primers to each vessel, but a different terminating nucleotide and carry out the extension in the presence of all four dNTPs, where each terminating nucleotide is differently labeled. After completion of the extension, one could combine the product from the four vessels and read the sequence by the mobility of the different extended primers, detecting the terminating nucleotide by the different labels. Alternatively, one could have the extension reaction carried out in a single vessel with all four terminating nucleotides present.

The subject invention greatly simplifies carrying out highly multiplexed reactions in one or a few vessels, and greatly simplifies highly multiplexed analyses of many reactions. By having two different variables, the capture oligonucleotide and an identifier, greater flexibility is obtained in the choice of the identifier and one can provide for sharper differentiation in the detection of the different identifiers. Also, the number of different molecules required to be synthesized is reduced, since one may employ a smaller repertoire of identifiers, while still achieving the required diversity for the identification of the individual events.

It is evident that the subject method provides for great versatility in scoring a large number of events in a reliable and accurate manner. By using a combination of varying oligonucleotides which may be individually released with a repertoire of identifiers, one is able to multiply the number of identifiers with the number of varying oligonucleotides, greatly enhancing the number of individual events which may be scored, while still permitting a simple analytical method. Since the number of molecules, which must be assayed in one determination, is only a fraction of the total number of events to be determined, one can provide for substantial distinctions between the identifiers, enhancing reliability and accuracy in scoring the events.

All publications, patents and patent applications mentioned in this specification are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for separating nucleic acid reagents into subsets wherein nucleic acid reagents are captured by hybridization and selectively released, using a sequestering agent and a plurality of pairs of nucleic acid reagents and capture reagents, wherein each nucleic acid reagent comprises a sequence part for hybridizing to a capture reagent, and said capture reagents comprise a complementary sequence to each of said sequence parts; said method comprising:
(a) combining said sequestering agent, said nucleic acid reagents and said capture reagents, whereby said nucleic acid reagents form hybrids with said capture reagents, and said hybrids are sequestered by said sequestering agent;
(b) separating the reagent medium from said sequestering agent; and
(c) releasing sequentially subsets of said nucleic acid reagents from said sequestering agent by sequentially increasing stepwise the stringency conditions, to provide said separated subsets.

2. A method according to claim 1, wherein said capture reagents are sequestered by said sequestering agent prior to use in said combining step.

3. A method according to claim 1, wherein said stringency is increased by varying at least one of the temperature, buffer concentration, salt concentration, pH, electric field, and organic co-solvent concentration.

4. A method for performing multiplexed determinations of target nucleic acid where primer reagents are modified, captured by hybridization and selectively released, using a plurality of pairs of primer reagents and capture reagents, wherein each primer reagent comprises a first sequence part for hybridizing to a target and a second sequence part for hybridizing to a capture reagent, and said capture reagents comprise a complementary sequence to each of said second sequence parts, a sequestering agent, and wherein an enzymatic reagent system is employed for modifying primer reagent bound to target nucleic acid, said method comprising:
(a) combining target nucleic acid with said primer reagents, said enzymatic reagent system, said capture reagents and said sequestering agent, whereby primer reagent bound to target nucleic acid is modified, said primer reagents form hybrids with said capture reagents, and said hybrids are sequestered onto said sequestering agent;
(b) separating other components from said sequestering agent;
(c) releasing sequentially subsets of said primer reagents from said sequestering agent by sequentially increasing stepwise the stringency conditions; and
(d) analyzing said primer reagents of each sequentially released subset, to provide said multiplexed determination.

5. A method according to claim 4, wherein said capture reagents are sequestered onto said sequestering agent prior to use in said combining step.

6. A method according to claim 4, wherein said stringency is increased by varying at least one of the temperature, buffer concentration, salt concentration, pH, electric field, and organic co-solvent concentration.

7. A method according to claim 4, wherein said analyzing is by electrophoresis.

8. A method for performing multiplexed determinations of target nucleic acid where primer reagents are modified, captured by hybridization and selectively released, using (1) a plurality of pairs of primer reagents and capture reagents, wherein (a) each primer reagent comprises a first sequence part for hybridizing to a target and a second sequence part for hybridizing to a capture reagent, and (b) said capture reagents comprise a complementary sequence to each of said second sequence parts, wherein during the release process the captured primer reagents to be released have a melting temperature at least 10° C. lower than the other captured primer reagents; (2) a sequestering means; and (3) wherein an enzymatic reagent system is employed for modifying primer reagent bound to target nucleic acid, said method comprising:
(a) combining target nucleic acid with said primer reagents, said enzymatic reagent system, said capture reagents and said sequestering agent, whereby primer reagent bound to target nucleic acid is modified in length by at least one nucleotide, said primer reagents form hybrids with said capture reagents, and said hybrids are sequestered onto said sequestering agent;
(b) separating other components from said sequestering agent;
(c) releasing sequentially subsets of said primer reagents from said sequestering agent by sequentially increasing stepwise the stringency conditions; and
(d) analyzing said primer reagents in each sequentially released subset, to provide said multiplexed determination.

9. A method according to claim 8, wherein said releasing of each subset occurs by increasing the temperature stepwise in 10° C. increments.

10. A method according to claim 8, wherein said releasing of each subset occurs by increasing the stringency by varying one or both of the salt concentration and the organic co-solvent concentration.

11. A method according to claim 8, wherein said releasing of each subset occurs by increasing the stringency by increasing the temperature stepwise in increments less than 10° C. while varying one or both of the salt concentration and the organic co-solvent concentration.

12. A method according to claim 8, wherein said capture reagents are sequestered onto said sequestering agent prior to use in said combining step.

13. A method according to claim 8, wherein in said combining step said capture reagents and said primer reagents form hybrids prior to sequestering said hybrids onto said sequestering agent.

14. A method according to claim 8, wherein said primer reagents have a non-replicable moiety or junction between said first sequence part and said second sequence part.

15. A method according to claim 8, wherein said primer reagents are further comprised of an identifier identifying said primer.

16. A method according to claim 15, wherein said primer oligonucleotides have different mobilities based on different identifiers.

17. A method according to claim 8, wherein said modifying of said primer reagent bound to target is an extension by at least one nucleotide, and wherein said at least one nucleotide in said extension comprises a labeled terminating nucleotide.

18. A method according to claim 17, wherein said modifying by extension is performed in four different vessels, each vessel having a different terminating nucleotide and one of said primer reagent or said terminating nucleotide is differently labeled to identify said terminated extended primer.

19. A method for performing multiplexed determinations of target nucleic acid where primer reagents are modified, captured by hybridization and selectively released, using (1) a plurality of pairs of primer reagents and capture reagents, wherein (a) each primer reagent comprises a first sequence part for hybridizing to a target and a second sequence part for hybridizing to a capture reagent, and (b) said capture reagents comprise a complementary sequence to each of said second sequence parts, wherein during the release process the captured primer reagents to be released have a melting temperature at least 10° C. lower than the other captured primer reagents, with the proviso that there is at least one group comprising a plurality of either primer reagents or capture reagents that further comprise a site for modification by a strand cleaving reagent system; (2) a sequestering means; (3) wherein an enzymatic reagent system is employed for modifying primer reagent bound to target nucleic acid; and (4) wherein a different strand cleaving reagent system is employed for modifying each of said at least one group of primer reagents or capture reagents; said method comprising:
(a) combining target nucleic acid with said primer reagents, said enzymatic reagent system, said capture reagents and said sequestering agent, whereby primer reagent bound to target nucleic acid is modified in length by at least one nucleotide, said primer reagents form hybrids with said capture reagents, and said hybrids are sequestered onto said sequestering agent;
(b) separating other components from said sequestering agent;
(c) releasing sequentially subsets of said primer reagents from said sequestering agent by sequentially increasing stepwise the stringency conditions;
(d) combining a strand cleaving reagent system with said sequestered primer reagents, whereby one of said at least one group of capture reagents or primer reagents are modified;
(e) releasing sequentially subsets of said primer reagents from said sequestering agent by sequentially increasing stepwise the stringency conditions;
(f) repeating said combining and releasing steps (d) and (e) until all desired primer reagents have been released; and
(g) analyzing said primer reagents in each sequentially released subset, to provide said multiplexed determination.

20. A method according to claim 19, wherein said strand cleaving reagent system is a restriction enzyme system.

21. A method according to claim 19, wherein said releasing steps (c) and (e) occur by increasing the temperature stepwise in 10° C. increments.

22. A method according to claim 19, wherein said releasing steps (c) and (e) occur by increasing the stringency by varying one or both of the salt concentration and the organic co-solvent concentration.

23. A method according to claim 19, wherein said releasing steps (c) and (e) occur by increasing the stringency by increasing the temperature stepwise in increments less than 10° C. while varying one or both of the salt concentration and the organic co-solvent concentration.

24. A method according to claim 19, wherein said capture reagents are sequestered onto said sequestering agent prior to use in said combining step.

25. A method according to claim 19, wherein in said combining step said capture reagents and said primer reagents form hybrids prior to sequestering said hybrids onto said sequestering agent.

26. A method according to claim 19, wherein said primer reagents are further comprised of an identifier identifying said primer.

27. A method for performing multiplexed determinations of target DNA to determine a multiplicity of greater than about 50 genotypes at single positions, using a combination of reagents, including: (1) primer reagents having (a) a first sequence part homologous to a target nucleic acid sequence wherein the 3′ terminal nucleotide is adjacent to a single base position of interest; (b) a second sequence part having a sequence homologous to a capture reagent; and (c) an identifier moiety identifying said primer oligonucleotide, wherein said primer reagents have at least three different second sequence parts; (2) capture reagents homologous to said different second sequence parts, wherein during the release process the captured primer reagents to be released have a melting temperature at least 10° C. lower than the other captured primer reagents; and (3) a sequestering agent; said method comprising:
(a) combining under hybridizing conditions said target DNA and said primer reagents, whereby said primers hybridize to homologous sequences present in said target DNA to form primer duplexes;
(b) extending said primers in said primer duplexes with a polymerase to add one terminating nucleotide to said primer reagent complementary to the DNA target sequence to form extended primer sequences;
(c) dissociating said extended primer sequences from homologous sequences;
(d) repeating steps (a), (b) and (c) to produce additional extended primer sequences;
(e) combining said extended primer sequences, said capture reagents and said sequestering agent, whereby said primer reagents form hybrids with said capture reagents and said hybrids are sequestered onto said sequestering agent;
(f) releasing sequentially subsets of said primer reagents from said sequestering agent by sequentially increasing stepwise the stringency conditions; and
(g) analyzing said extended primers by means of said identifier moieties to provide said multiplexed genotype determination.

28. A method according to claim 27, wherein it is further provided that there is at least one group comprising a plurality of either said primer reagents or said capture reagents that further comprise a site for modification by a strand cleaving reagent system; and wherein a different strand cleaving reagent system is employed for modifying each of said at least one group of primer reagents or capture reagents; wherein following step (f), said method further comprises the steps of:
(h) combining a strand cleaving reagent system with said sequestered primer reagents, whereby one of said at least one group of capture reagents or primer reagents are modified;
(i) releasing sequentially subsets of said primer reagents from said sequestering agent by sequentially increasing stepwise the stringency conditions;
(j) repeating said combining and releasing steps (h) and (i) until all desired primer reagents have been released; and
said step (g).

29. A method according to claim 27, wherein said extending is performed in four different vessels, each vessel having a different terminating nucleotide and one of said primer reagent or said terminating nucleotide is differently labeled to identify said terminating nucleotide.

30. A method according to claim 27, wherein said extending is performed in a single vessel with four different terminating nucleotides, each terminating nucleotide labeled with a different label to identify said terminating nucleotide.

31. A method according to claim 27, wherein said identifier is a mobility tag.

32. A method according to claim 31, wherein said mobility tag is for electrophoretic separations.

33. A method according to claim 32, wherein said mobility tag is comprised of different length nucleic acid sequences.

34. A method for performing sequencing of target DNA comprising at least about 2 kb to determine the sequence of said target DNA, using a combination of reagents, including: (1) primer reagents having (a) a first sequence part homologous to a target nucleic acid sequence and (b) a second sequence part homologous to a capture reagent, wherein said primer reagents have at least three different second sequence parts, with the proviso that said first part can serve as the first and second parts; (2) capture reagents homologous to said different second sequence parts, wherein during the release process the captured primer reagents to be released have a melting temperature at least 10° C. lower than the other captured primer reagents; (3) a sequestering agent; and (4) a template-dependent extension terminator; said method comprising:
(a) combining under hybridizing conditions said target DNA and said primer reagents, whereby said primers hybridize to homologous sequences present in said target DNA to form primer duplexes;
(b) extending said primers in said primer duplexes with a polymerase in the presence of dNTPs and at least one terminator nucleotide, to add said dNTPs and said at least one terminator nucleotide to said primer reagent to extend said primer reagent with a sequence complementary to the DNA target sequence to form extended primer sequences, with the proviso that the extending will include at least two different terminator nucleotides in the same or different vessels;
(c) dissociating said extended primer sequences from homologous sequences;
(d) repeating steps (a), (b) and (c) to produce additional extended primer sequences;
(e) combining said extended primer sequences, said capture reagents and said sequestering agent, whereby said primer reagents form hybrids with said capture reagents and said hybrids are sequestered onto said sequestering agent;
(f) releasing sequentially subsets of said primer reagents from said sequestering agent by sequentially increasing stepwise the stringency conditions; and
(g) analyzing said extended primers to determine the sequence of said target DNA.

35. A method according to claim 34, wherein it is further provided that there is at least one group comprising a plurality of either said primer reagents or said capture reagents that further comprise a site for modification by a strand cleaving reagent system; and wherein a different strand cleaving reagent system is employed for modifying each of said at least one group of primer reagents or capture reagents; wherein following step (f), said method further comprises the steps of:
(h) combining a strand cleaving reagent system with said sequestered primer reagents, whereby one of said at least one group of capture reagents or primer reagents are modified;
(i) releasing sequentially subsets of said primer reagents from said sequestering agent by sequentially increasing stepwise the stringency conditions;
(j) repeating said combining and releasing steps (h) and (i) until all desired primer reagents have been released; and
said step (g).

36. A method according to claim 34, wherein said target DNA derives from one contiguous strand.

37. A method according to claim 34 wherein said target DNA derives from at least two strand fragments, plasmids or vectors.

38. A method according to claim 34, wherein said extending is performed in four different vessels, each vessel having a different terminating nucleotide and one of said primer reagent or said terminating nucleotide is differently labeled to identify said terminating nucleotide.

39. A method according to claim 34, wherein said extending is performed in a single vessel with four different terminating nucleotides, each terminating nucleotide labeled with a different label to identify said terminating nucleotide.

40. A method for multiplexing the sequencing of target DNA using a combination of reagents including (1) at least three primer reagents having (a) a first sequence part homologous to a target nucleic acid sequence, and (b) a second sequence part which comprises said first sequence part; (2) capture reagents homologous to said different second sequence parts and comprising a sequestering means, wherein during the release process the captured primer reagents to be released have a melting temperature at least 10° C. lower than any other captured primer reagents; and (3) a template dependent extension terminator; with the proviso that one of said primer reagents or said extension terminator is labeled with a detectable label, said method comprising:
(a) combining said target DNA, said primer reagent and said template dependent extension terminator under conditions for hybridization and chain extension, whereby said primer oligonucleotides hybridize to and are extended along said target DNA to form extended primers hybridized to said target DNA;
(b) dissociating said extended primers from said target DNA;
(c) repeating said combining and dissociating steps to provide sufficient numbers of extended primers for sequencing of said target DNA;
(d) hybridizing said primer reagents to capture reagents sequestered onto a sequestering agent;
(e) releasing sequentially subsets of said primer reagents from said sequestering agent by sequentially increasing stepwise the stringency conditions; and
(f) analyzing said extended primers to provide said multiplexed sequencing.

41. A kit comprising the separate components of: (1) a set of primer reagents having (a) a first sequence part homologous to a target nucleic acid sequence, (b) a second sequence part having a sequence homologous to a capture reagent, wherein said primer reagents have at least three different said second sequence parts; (2) capture reagents homologous to said different second sequence parts; wherein the hybrids formed by said primer reagents and said capture reagents are subject to sequential release under conditions of sequential stepwise increased stringency.

42. A kit according to claim 41, further comprising a sequestering agent.

43. A kit according to claim 41, wherein said capture reagents are provided sequestered onto a sequestering agent.

44. A kit according to claim 41, further comprising an enzymatic reagent system for modifying said primer reagents.

45. A kit according to claim 41, wherein said primer reagents further comprise an identifier identifying each of said primer reagents.

46. A kit according to claim 38, wherein said primer reagents are provided in four separate containers with four different labels for use in separate vessels.

47. A kit according to claim 41, wherein at least one group comprising a plurality of said primer reagents further comprise a site for modification by a strand cleaving reagent system, whereby upon modification said group of primer reagents are subject to sequential release under conditions of sequential stepwise increased stringency.

48. A kit according to claim 47, further comprising at least one strand cleaving reagent system.
